(19) United States
(12) Patent Application Publication
(10) Pub. No.: US 2020/0390352 A1
(43) Pub. Date: Dec. 17, 2020

(54) SAFETY LOCKOUT FOR PHOTOPLETHYSMOGRAM SENSOR

(71) Applicant: Medic, Inc., Provo, UT (US)

(72) Inventors: David R. Hall, Provo, UT (US); K. Jeffrey Campbell, Spanish Fork, UT (US); Alec Raymond, Orem, TX (US)

(21) Appl. No.: 16/889,561

(22) Filed: Jun. 1, 2020

Related U.S. Application Data
(60) Provisional application No. 62/862,659, filed on Jun. 17, 2019.

(51) Int. Cl. A61B 5/024

(52) U.S. Cl. CPC A61B 5/02427

(57) ABSTRACT

A photoplethysmography (PPG) sensor and methods for safely operating a PPG sensor are described. The sensor includes a PPG light source (e.g., a light emitting diode (LED), a laser), a photo detector, a memory, and a processor in electronic communication with the memory. The memory stores instructions that when executed by the processor cause the processor to detect a light level via the photo detector, determine an ambient light level based on the detected light level, determine that the photo detector is obstructed when the ambient level satisfies a first threshold, operate the PPG light source at a first level when the photo detector is unobstructed, and operate the PPG light source at a second level when the photo detector is obstructed.

### CROSS-REFERENCE TO RELATED APPLICATIONS§

This application claims the benefit of U.S. Provisional Application No. 62/862,659, filed Jun. 17, 2019, which is incorporated by reference herein in its entirety.

### TECHNICAL FIELD

This application is related to sensors that emit light as part of the sensing operation. In particular, this application is related to photoplethysmogram (PPG) sensors.

### BACKGROUND

Photoplethysmogram (PPG) sensors operate by emitting photons through tissue and detecting attributes associated with the tissue based on how the photons are absorbed as they travel through the tissue. The intensity of the photons emitted by the light source of a PPG sensor can be obnoxious and/or harmful to human eyes. Often shields or shrouds are used to contain/enclose the emitted photons. However, depending on the use/placement of PPG sensors, shields and/or shrouds may not be practically used. Accordingly, there is a need for solutions that enable PPG sensors to be used without enclosing the PPG sensor with shields and/or shrouds.

### SUMMARY

In a first aspect, the disclosure provides a sensor. The sensor includes a photoplethysmography (PPG) light source (e.g., a light emitting diode (LED), a laser), a photo detector, a memory, and a processor in electronic communication with the memory. The memory stores instructions that when executed by the processor cause the processor to: detect a light level via the photo detector, determine an ambient light level based on the detected light level, determine that the photo detector is obstructed when the ambient level satisfies a first threshold, operate the PPG light source at a first level when the photo detector is unobstructed, and operate the PPG light source at a second level when the photo detector is obstructed.

In a second aspect, the disclosure provides that the instructions are further executable by the processor to determine that the photo detector is unobstructed when the ambient light level exceeds the first threshold.

In a third aspect, the disclosure provides that the PPG light source, when operated at the second level, emits photons with sufficient energy to generate PPG signals from human tissue, that the PPG signals are generated by the emitted photons that are selectively and characteristically reflected by the human tissue, and that the human tissue is leg tissue.

In a fourth aspect, the disclosure provides that the photo detector receives the PPG signals reflected by the human tissue.

In a fifth aspect, the disclosure provides that the PPG light source, when operated at the first level, emits no photons. That is, the PPG light source is off when operated at the first level.

In a sixth aspect, the disclosure provides that the PPG light source, when operated at the first level, emits photons at a safe energy level or emits photons at an unsafe energy level for short enough durations of time to render the emitted photons safe to enter a human eye without causing harm to the human eye. That is, the PPG light source emits photons that are safe for the human eye when operated at the first level.

In a seventh aspect, the disclosure provides that the PPG light source, when operated at the first level, emits photons that enable self-diagnostic of the PPG sensor, and that the PPG light source, when operated at the first level, emits photons that enable calibration of the PPG sensor.

In an eighth aspect, the disclosure provides that the ambient light substantially provided by the PPG light source when operated at the first level.

In a ninth aspect, the disclosure provides that the PPG light source is at least one of a light emitting diode and a laser.

In a tenth aspect, the disclosure provides that the photo detector is one of a photo diode, a photo transistor, and a photo resistor.

In an eleventh aspect, the disclosure provides a method for operating an unsheathed/enshrouded photoplethysmography (PPG) sensor. The method includes detecting a light level via a photo detector, determining an ambient light level based on the detected light level, determining that the photo detector is obstructed when the ambient light level satisfies a first threshold, operating a PPG light source at a first level when the photo detector is unobstructed, and operating the PPG light source at a second level when the photo detector is obstructed.

In a twelfth aspect, the disclosure provides that the method further includes determining that the photo detector is unobstructed when the ambient light level exceeds the first threshold.

In a thirteenth aspect, the disclosure provides that operating the PPG light source at the second level comprises emitting photons with sufficient energy to generate PPG signals from human tissue, that the PPG signals are generated by emitting photons that are selectively and characteristically reflected by the human tissue, and that the human tissue is leg tissue.

In a fourteenth aspect, the disclosure provides that The method of claim 13, further comprising receiving PPG signals via the photo detector, wherein the PPG signals are reflected photons emitted by the PPG light source, and wherein the emitted photons are reflected by the human tissue.

In a fifteenth aspect, the disclosure provides that operating the light source at the first level comprises emitting no photons. That is, the PPG light source is off when operating the PPG light source at the first level.

In a sixteenth aspect, the disclosure provides that operating the PPG light source at the first level comprises emitting photons that are safe to enter a human eye without causing harm to the human eye, and that emitting photons that are safe to enter the human eye comprises emitting photons at a safe energy level or emitting photons at an unsafe energy level for short enough durations of time to render the emitted photons safe to enter a human eye.

In a seventeenth aspect, the disclosure provides that operating the PPG light source at the first level comprises emitting photons that enable self-diagnostic of the PPG sensor, and that operating the PPG light source at the first level comprises emitting photons that enable calibration of the PPG sensor.

In an eighteenth aspect, the disclosure provides that the ambient light level is substantially provided by operating the PPG light source at the first level.

In a nineteenth aspect, the disclosure provides that the PPG light source is at least one of a light emitting diode and a laser.

In a twentieth aspect, the disclosure provides that the photo detector is one of a photo diode, a photo transistor, and a photo resistor.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a perspective diagram illustrating an exemplary embodiment of a toilet in which the described systems, methods, and devices may be implemented.

FIG. 2 is a perspective diagram illustrating a top-down perspective of the toilet illustrated in FIG. 1.

FIG. 3 is an illustrative diagram of an environment in which a PPG sensor is unobstructed and operating in an inactive mode.

FIG. 4 is an illustrative diagram of an environment in which a PPG sensor is unobstructed and operating in an inactive mode.

FIG. 5 is a perspective diagram illustrating a side view perspective of a user sitting on the toilet.

FIG. 6 is an illustrative diagram f an environment in which a PPG sensor is obstructed while operating in an inactive mode.

FIG. 7 is an illustrative diagram f an environment in which a PPG sensor is obstructed while operating in an inactive mode.

FIG. 8 is an illustrative diagram of an environment in which a PPG sensor is obstructed and operating in a PPG measurement mode.

FIG. 9 is a flow diagram illustrating one example of a method for safely operating a PPG sensor.

FIG. 10 is a block diagram of a computing device for implementing the described systems, methods, and devices.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

Some sensors combine a photo (e.g., light) emitting device with a sensing device to perform a particular sensing function. A prime example of this type of sensor is a photoplethysmogram (PPG) sensor. A PPG sensor includes a photo emitting device (e.g., light emitting diode (LED), laser) and a photo sensing device (e.g., photodiode, photo resistor, phototransistor, photosensor) that senses received photons (e.g., light). As light travels through tissue, which is composed of different biological tissues, it is absorbed differently by different types of biological tissue (e.g., bones, skin pigments, and both venous and arterial blood. For example, as light travels through tissue it is more strongly absorbed by both venous and arterial blood, which allows changes in blood flow to be detected by changes in intensity of light. A PPG sensor detects these changes in intensity of light (e.g., PPG waveform, plethysmograph) via the light sensing device.

In order to obtain proper PPG measurements, the photo emitting device must emit photons with sufficient intensity to ensure that the resulting PPG waveform is detectable/readable by the photo sensing device. The intensity of photons emitted by the photo emitting device, which is often in the infrared and/or the visible spectrum, may be obnoxious and or may be harmful (e.g., cause damage) to eyes (e.g., human eyes). Because of these challenges PPG sensors are often enclosed and/or shielded/shrouded to ensure that the photons emitted by the photo emitting device is blocked and/or prevented from entering the eyes of those nearby. For example, finger-based PPG sensors are generally shielded/shrouded, with a hinged, clamshell-type design, so that the shield/shroud/housing encloses over the finger to block and/or prevent any photon exposure outside of the enclosure.

It is appreciated that the photon intensity required for PPG measurements depends on which tissue in the body the PPG measurement is being performed on. For example, blood vessels are closer to the surface of the skin in the pads of the fingers than in the thighs of the legs. Accordingly, the photon intensity required for taking PPG measurements from a leg are higher than the photon intensity required for taking PPG measurement from a finger. Therefore, the challenge of obnoxious or harmful photon emissions from the photo emitting device may be exaggerated depending upon the type of tissue that the PPG sensor is designed to measure.

It is further appreciated that the traditional approach of enclosing and/or shielding the photo emitting device may be impractical in certain use cases (e.g., taking PPG measurements from tissue of the body that is not in the fingers (e.g., legs, feet, arms, wrists, etc.)). Therefore, there is a need to enable PPG measurements of these different areas of tissues while ensuring that photon emissions from the photo emitting device are not obnoxious nor harmful to others.

The described systems, methods, and devices determines whether the PPG sensor is obstructed and adjusts (i.e., decreases) the photon intensity emitted by the photo emitting device to ensure that no photons are emitted (e.g., the light is turned off) or photons are emitted at a level that is neither obnoxious nor harmful (e.g., the intensity of the light is dimmed). When it is determined that the PPG sensor is obstructed, the photon intensity emitted by the light emitting device may be adjusted (i.e., increased) to the required levels for PPG measurements. Accordingly, the PPG sensor may be effectively used without the need for shields or enclosures.

The following description describes the use of a PPG sensor in a toilet seat. It is appreciated that the described systems, methods, and devices enable a PPG sensor to be integrated into a toilet seat. It is further appreciated that the described systems, methods, and devices similarly enable a PPG sensor to be integrated into any of a variety of devices (e.g., a floormat (i.e., for PPG sensor measurements from a foot), a seat (i.e., as in the case of the toilet seat), backrest (i.e., for PPG sensor measurements from a back), etc.).

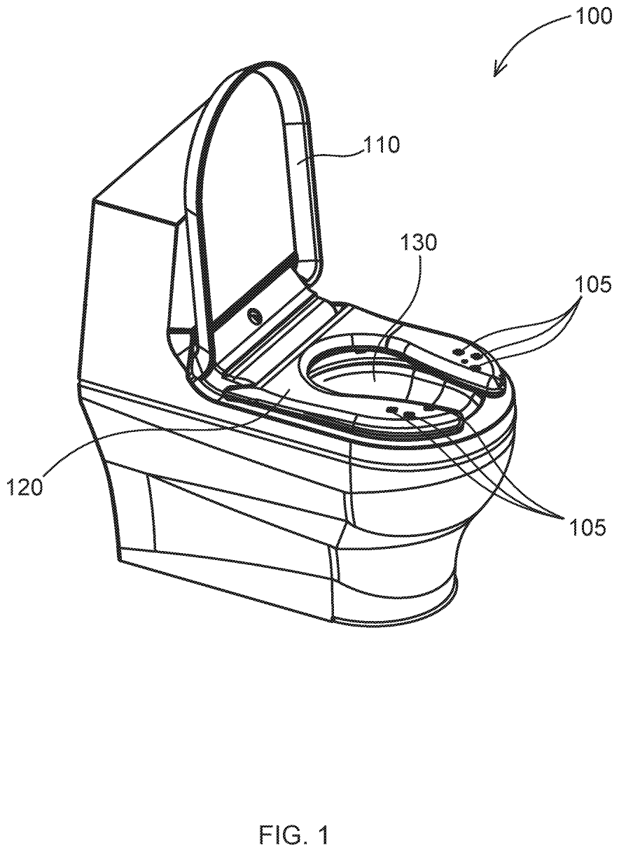

FIG. 1 is a perspective diagram illustrating an exemplary embodiment of a toilet 100 in which the described systems, methods, and devices may be implemented. The toilet 100 includes a toilet bowl 130 (for receiving excreta, for example) as well as a toilet seat 120 and a toilet lid 110 that are positioned above the toilet bowl 130. The toilet seat 120 provides a resting place for a user to sit on while providing access for excreta to enter the toilet bowl 130. The toilet lid 110 may substantially cover the toilet seat 120 and/or the toilet bowl 130 so that the toilet is more aesthetically pleasing when not in use.

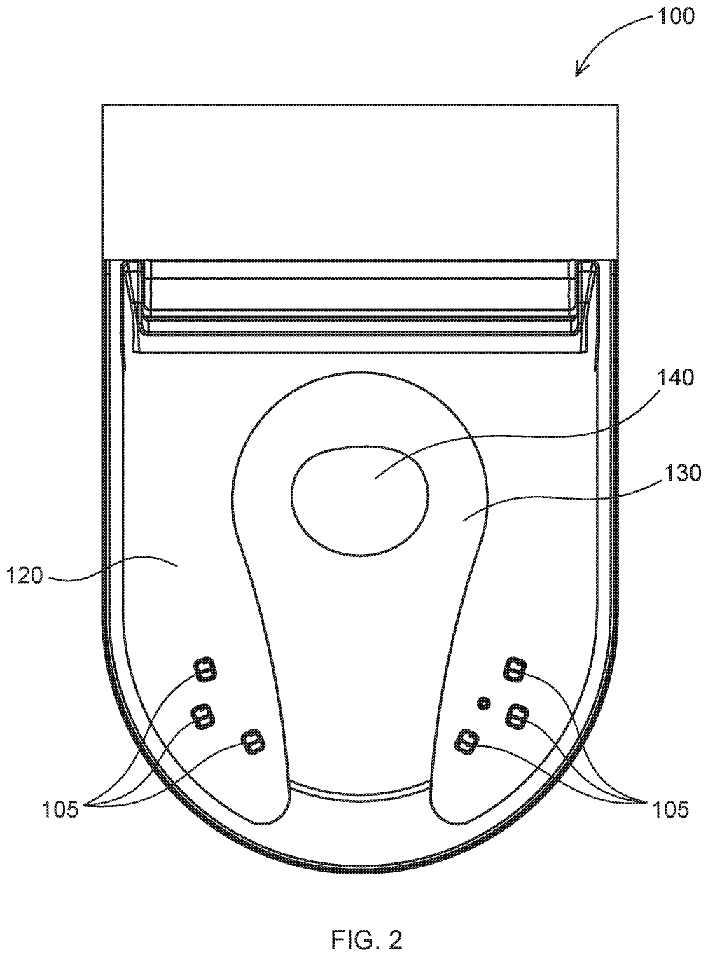

The toilet seat 120 includes one or more PPG sensors 105 integrated into the toilet seat 120. The PPG sensors 105 may be strategically positioned in the toilet seat 120 to be located under the thigh (e.g., hamstring area) of a user when the user is sitting on the toilet seat 120. In some cases, there may be multiple PPG sensors 105 so that at least one can provide good PPG sensor measurements when a user is sitting on the toilet seat 120. The placement of the PPG sensors 105 is illustrated more clearly in FIG. 2.

FIG. 2 is a perspective diagram illustrating a top-down perspective of the toilet 100 illustrated in FIG. 1. As illustrated in FIG. 2, the toilet bowl 130 includes an output 140 (which enables evacuating the contents of the toilet bowl 130, via flushing the toilet 100, for example). As illustrated, the toilet seat 120 is designed to comfortably support a user in a sitting position with the user's legs being directly over at least one of the PPG sensors 105.

A PPG sensor 105 is typically used for measurements on human subjects. A PPG sensor 105 includes a photo emitting device and a photo sensing device. These two devices (i.e., the photo emitting device and the photo sensing device) are housed in a housing that separates the photo emitting device from the photo sensing device (illustrated by the bisecting line in each PPG sensor 105, for example).

As described herein, the PPG sensor 105 (i.e., the photo sensing device of the PPG sensor 105), is repurposed to take preliminary photo (e.g., light) measurements to ensure safe operating condition exists before engaging (or fully engaging) the photo emitting device of the PPG sensor 105. The preliminary light measurements may be used to determine an ambient light level. Changes in the ambient light level may be used to detect that an obstruction is obstructing the PPG sensor 105 (i.e., the photo sensing device of the PPG sensor 105). The detection of an obstruction above the PPG sensors may be used as part of a safety lockout mechanism that ensures that the photo emitting device (e.g., LEDs, lasers) do not turn on unless they are blocked by an obstruction. Accordingly, this safety lockout mechanism ensures that emitted photons will not accidentally shine into a user's eye.

The photo sensing device (e.g., photo diode, photo resistor) detects photo (e.g., light) intensity. When the safety lockout is enabled (e.g., when the photo emitting device is inactive turned off or turned down), the photo sensing device detects ambient light. On the other hand, when the safety lockout is disabled (e.g., when an obstruction is detected and the photo emitting device is active/operating at PPG measurement levels), the photo sensing device detects reflected photons (e.g., photo intensities that represent PPG measurements).

In some embodiments the photo sensing device may perform both the ambient light level detection and the PPG measurement sensing. In other embodiments, a dedicated (i.e., separate, second) photo detector (e.g., a photodiode, photoresistor) may detect the ambient light level to enable the safety lockout, while the photo sensing device performs the PPG sensor measurements. In the case of a dedicated photo detector, the dedicated photo detector may be built into or adjacent to the photo sensing device.

The photo sensing device and/or the photo detector may be mounted above the plane of the photo emitting device, or in a divider that blocks photons from the photo emitting device from directly impinging on the photo sensing device along pathways that do not result from reflections above the photo emitting device. While the photo detector may be a separate device than the photo sensing device, it is described herein as being a single device and is identified herein as the photo sensing device.

Background measurements from the photo sensing device, when the photo emitting device is off (e.g., is not emitting any photons), can be used to estimate whether there is any obstruction directly over or in direct contact with the PPG sensor 105. In an illuminated room an elevated background measurement (of the ambient light, for example) will often be sufficient to indicate that there is no obstruction present. In a dark room, an external light source (which provides an ambient light, for example) can be used to detect an obstruction above the PPG sensor 105. For example, an incandescent or LED installed for this purpose (or an ancillary purpose) elsewhere in the toilet 100 may provide ambient light for enabling obstruction detection.

In some embodiments, the photo emitting device in the PPG sensor 105 may be used at a safe level to induce elevated measurements (e.g., an ambient light scenario), which indicates that there is no user (e.g., obstruction) directly positioned on the toilet seat (e.g., above the PPG sensor 105). When the photo emitting device is used at a safe levels, the photo emission (e.g., illumination) can be held below a safe threshold, or held at a diagnostic threshold, or limited in duration in the absence of an obstruction to ensure safe operation when no obstruction is detected.

In some embodiments, photo emission or illumination limits may be imposed on all available PPG sensors 105 due to the measurement of one PPG sensor 105, or on a case by case basis. In this way, a PPG sensor group that is partially unobstructed will not prevent other PPG sensors 105 from taking measurements. In some cases, multiple lockout enabled photo sensor devices may be deployed within or in the vicinity of the PPG sensor 105 to improve functionality.

As noted above, similar light exposure problems exist if a PPG sensor 105 (e.g., light emitting device) is mounted on the back of the toilet lid, or in a weight scale placed on the floor in front of the toilet. In each case, it would be common for the user to come into direct exposure of obnoxious or harmful photons from a photo emitting device of a PPG sensor 105 that is actively functioning. The present described systems, methods, and devices are applicable to all unshrouded PPG sensors 105 installed in devices other than a toilet seat.

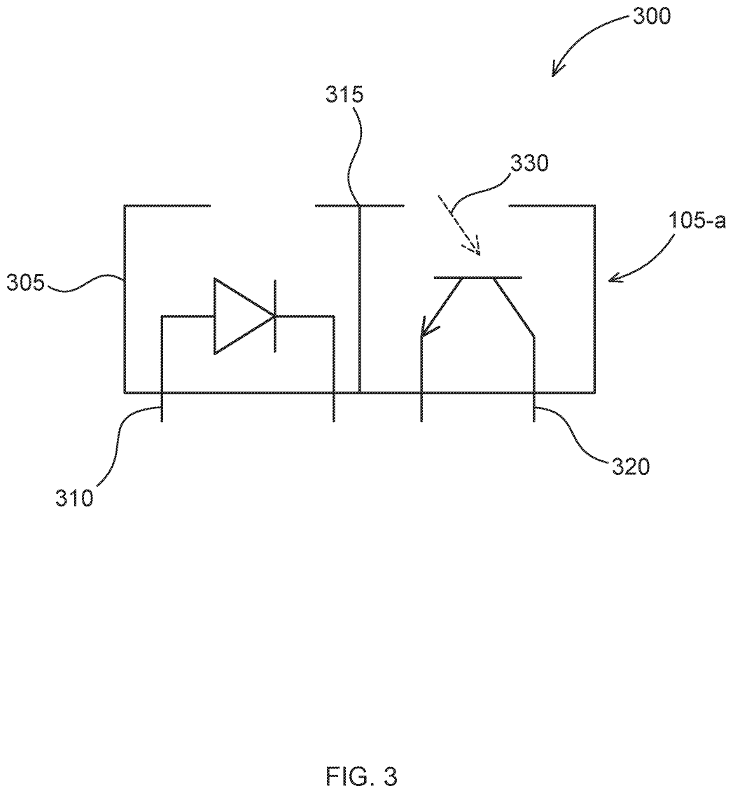

FIG. 3 is an illustrative diagram of an environment 300 in which a PPG sensor 105-a is unobstructed and operating in an inactive mode (e.g., safety lockout is enabled, not emitting any photons). The PPG sensor 105-a is an example of the PPG sensors 105 illustrated in FIGS. 1 and 2. The PPG sensor 105-a includes a photo emitting device 310 (as illustrated by the light emitting diode (LED) circuit symbol 310, for example) and a photo sensing device 320 (as illustrated by the phototransistor circuit symbol 320, for example). The photo emitting device 310 and the photo sensing device 320 are housed within a housing 305 that includes a divider 315 (e.g., barrier) that blocks photons from the photo emitting device 310 from directly impinging on the photo sensing device 320 along pathways that do not result from reflections from above the photo sensing device 320.

As illustrated in FIG. 3, the photo sensing device 320 is not obstructed and is receiving ambient light 330. Because the PPG sensor 105-a is not obstructed and as a result of the safety lockout, the photo emitting device 310 may be in an inactive mode (e.g., off/not emitting any photons, in this example). In this inactive mode, the photo emitting device 310 is operating at a safe level (e.g., off) since no obnoxious or harmful photons are being emitted (i.e., indeed, no photons are being emitted by the photo emitting device 310).

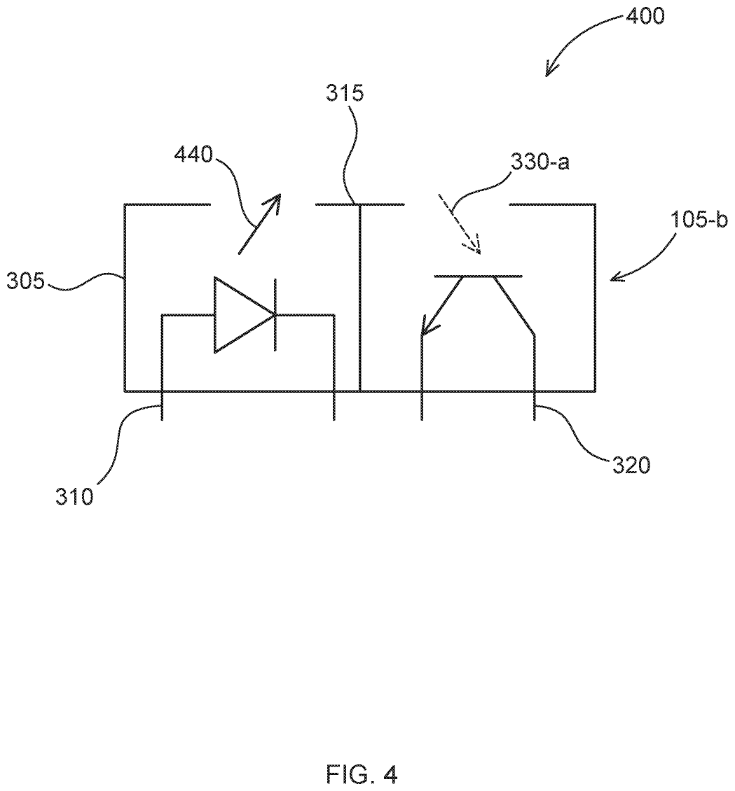

FIG. 4 is an illustrative diagram of an environment 400 in which a PPG sensor 105-b is unobstructed and operating in an inactive mode (e.g., safety lockout is enabled, emitting photons 440 at a safe level). The PPG sensor 105-b is an example of the PPG sensors 105 illustrated in FIGS. 1 and 2. As described with respect to FIG. 3, the PPG sensor 105-b includes a photo emitting device 310 and a photo sensing device 320 housed in a housing 305 that includes a divider 315.

As illustrated in FIG. 4, the photo sensing device 320 is not obstructed and is receiving ambient light 330-a. Because the photo sensing device 320 is not obstructed (the PPG sensor 105-b is not obstructed, for example), the photo emitting device 310 may be in an inactive mode (e.g., a safety lockout mode).

Alternatively, to emitting no photons in the inactive mode (as illustrated in FIG. 3, for example), the photo emitting device 310 may emit photons 440 at a safe level (as illustrated in FIG. 4, for example) when the photo emitting device 310 is in the inactive mode. This safe level is indicated by a single photon emission arrow 440. Emitting photons 440 at a safe level may include limiting the number and/or intensity of the photons that are being emitted by the photo emitting device 310. For example, the photo emitting device 310 may be operated at a reduced voltage and/or current to emit photons 440 within the safe level threshold (e.g., at a safe level). Additionally, or alternatively, the photo emitting device 310 may be operated at a reduced duty cycle (e.g., less than 20% duty cycle) using pulse width modulation, for example, to emit photons 440 within the safe level threshold. In this way, the photo emitting device 310 may emit photons 440 while still operating at a safe level (e.g., safe photon emission during the inactive mode) since the number and/or intensity of the of the emitted photons 440 are within the sale level threshold.

The ambient light 330-a is an example of the ambient light 330 illustrated in FIG. 3, except that the ambient light 330-a may include additional photons (e.g., ambient light) as a result of the safe level of photon emissions 440 from the photo emitting device 310. The ambient light level may be continually monitored to detect the presence of an obstruction.

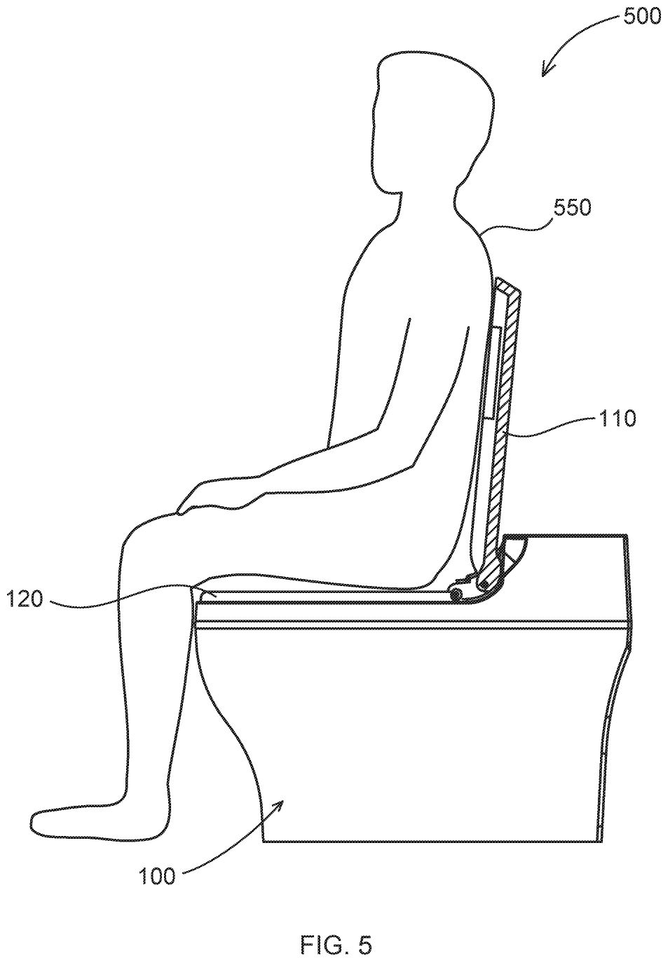

FIG. 5 is a perspective diagram illustrating a side viewperspective 500 of a user 550 sitting on the toilet 100. As described with respect to FIGS. 1 and 2, the toilet 100 includes a toilet seat 120 and a toilet lid. As illustrated in FIG. 5, the user 550 is sitting on the toilet seat 120 with the user's back being supported by the toilet lid 110. In this position, the user' legs obstruct one or more of the PPG sensors 105 illustrated in FIGS. 1 and 2.

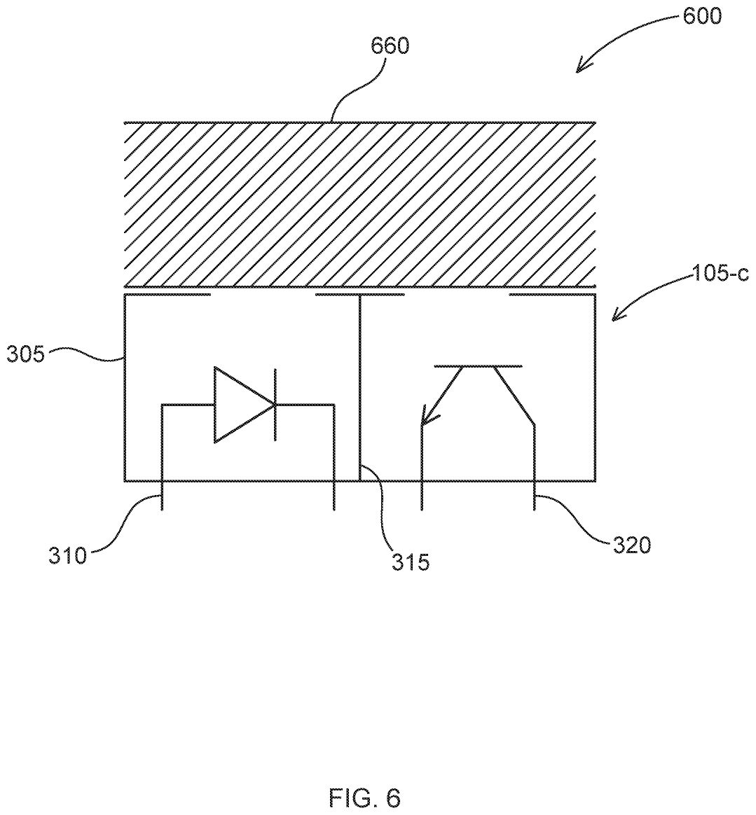

FIG. 6 is an illustrative diagram of an environment 600 in which a PPG sensor 105-c is obstructed while operating in an inactive mode (e.g., safety lockout is enabled, not emitting any photons). The PPG sensor 105-c is an example of the PPG sensor 105-a illustrated in FIG. 3.

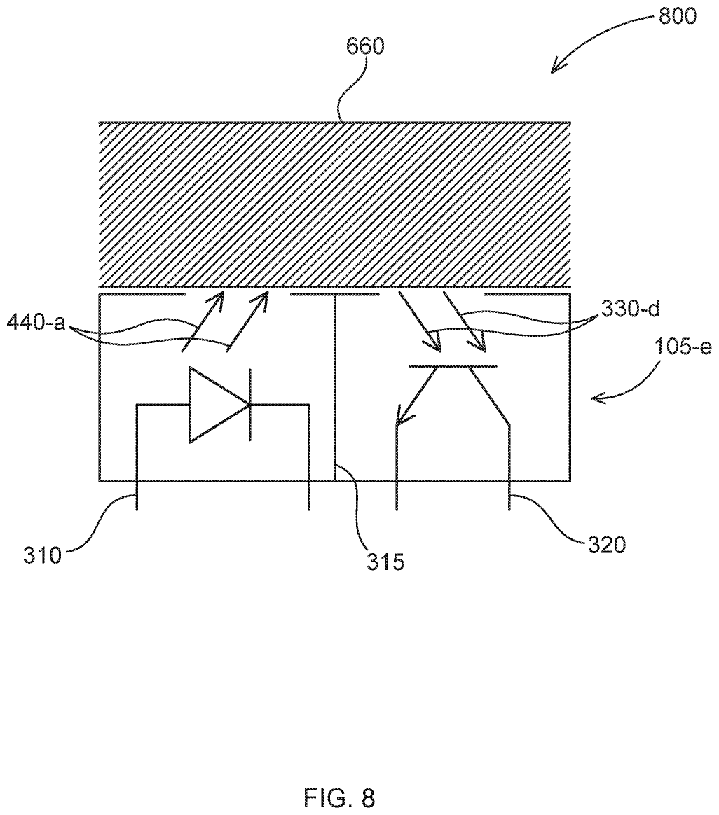

As illustrated in FIG. 6, the photo sensing device 320 (and the PPG sensor 105-c, for example) is obstructed by obstruction 660. In the context of FIG. 5, the obstruction 660 may be the leg of the user 550. As a result of the obstruction, the photo sensing device 320 is receiving little or no ambient light (e.g., ambient light 330) due to the obstruction. This substantial change in the amount of ambient light that is being received by the photo sensing device 320 triggers a change of the photo emitting device 310 from an inactive mode (e.g., safety lockout mode, as illustrated in FIG. 6, for example) to an active mode (e.g., PPG measurement mode, as illustrated in FIG. 8, for example).

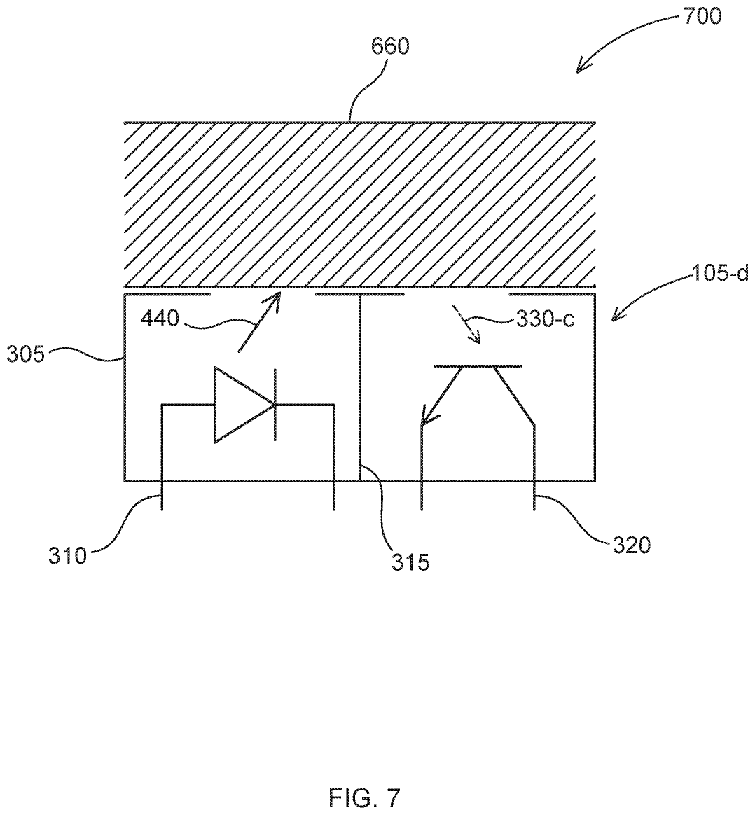

FIG. 7 is an illustrative diagram of an environment 700 in which a PPG sensor 105-d is obstructed while operating in an inactive mode (e.g., safety lockout is enabled, emitting photons 440 at a safe level). The PPG sensor 105-d is an example of the PPG sensor 105-b illustrated in FIG. 4.

As illustrated in FIG. 7, the photo sensing device 320 (and the PPG sensor 105-d, for example) is obstructed by obstruction 660. In the context of FIG. 5, the obstruction 660 may be the leg of the user 550. As a result of the obstruction 660, the photo sensing device 320 may receive some ambient light 330-c (a portion of ambient light 330-a, for example) due to emitted photons 440 being reflected by and/or passing through the obstruction 660. The change from ambient light 330-a to ambient light 330-c represents a substantial change in the amount of ambient light that is being received by the photo sensing device 320. When the change in ambient light exceeds a threshold, the change in ambient light is interpreted as an obstruction detection. The detection of an obstruction triggers a change of the photo emitting device 310 from an inactive mode (e.g., safety lockout mode, as illustrated in FIG. 7, for example) to an active mode (e.g., PPG measurement mode, as illustrated in FIG. 8, for example).

Although the ambient light 330-c may be reflected/passing photons as a result of the photons emitted 440 by the photo emitting device 310, the number and/or intensity of the photons in the emitted photons 440 and/or the resulting ambient light 330-c may be insufficient for PPG measurements. Accordingly, even if the ambient light 330-c is solely a result of the emitted photons 440, the ambient light 330-c may be interpreted to be ambient light 330-c and not PPG measurements (because the photons (i.e., ambient light 330-c) are unusable as PPG measurements, for example).

FIG. 8 is an illustrative diagram of an environment 800 in which a PPG sensor 105-e is obstructed and operating in a PPG measurement mode (e.g., active mode, after switching from an inactive mode to the active mode). In other words, the safety lockout is disabled, and the PPG sensor 105-e is operating as intended to take PPG measurements.

As illustrated in FIG. 8, the photo sensing device 320 (and the PPG sensor 105-e, for example) is obstructed by obstruction 660 and operating in a PPG measurement mode. As a result of being in the PPG measurement mode, the photo emitting device 310 may emit photons 440-a at a number and/or intensity sufficient to enable PPG measurements via the photons 330-d (photons 330-d of sufficient number and/or intensity that enable PPG measurements, indicated by two photon reception arrows 330-d, for example) received by the photo sensing device 320. The emitted photons 440-a (indicated by two photon emission arrows 440-a) may be at a number and/or intensity that would be obnoxious and/or harmful to human eyes, but because the PPG sensor 105-e is obstructed by obstruction 660, there is no risk that the emitted photons 440-a may enter human eyes. In this way, the PPG sensor 105-e may operate normally (e.g., in a PPG measurement mode) while the PPG sensor 105-e is obstructed and may operate in a safety lockout mode at all times when the PPG sensor 105-e is unobstructed.

It is appreciated that the level of ambient light is constantly monitored during both the inactive mode and the active mode (e.g., PPG measurement mode) to ensure that the safety lockout is engaged at all times when an obstruction is not detected. It is further appreciated that ambient light levels may be determined by averaging individual samples of detected photon levels over a period of time and that the change in ambient light levels between an obstructed state and an unobstructed state is readily determined. For instance, obstruction detection may require that photon detection levels remain below a predetermined threshold (e.g., a maximum of the small component (e.g., ambient light 330-c) resulting from the reflections and transmissions of photons emitted 440 at a safe level during an obstruction) for a predetermined period of time (e.g., 1 second).

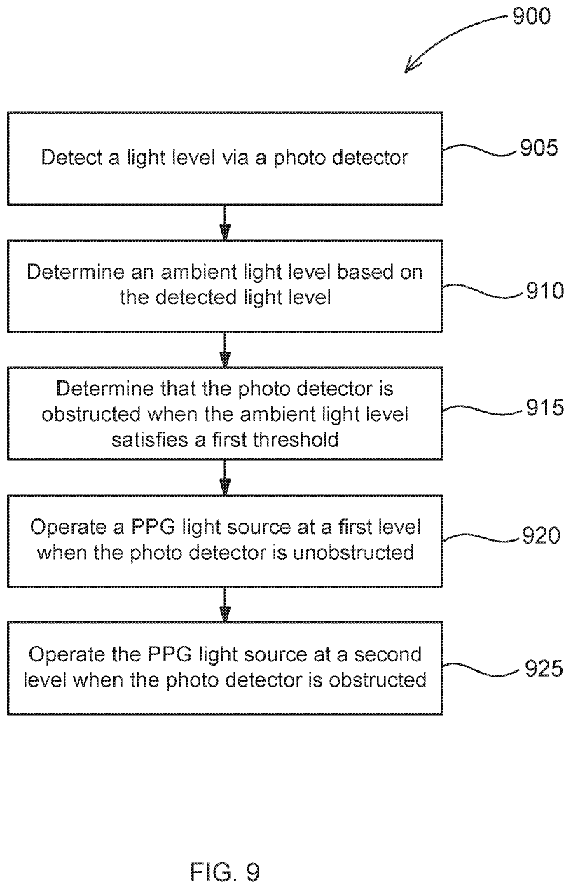

FIG. 9 is a flow diagram illustrating one example of a method 900 for safely operating a PPG sensor 105. The method 900 may be implemented by a PPG sensor 105 and more specifically by an application specific processor (e.g., processor and memory) included within the PPG sensor 105.

At 905, a light level (e.g., photon level) is detected via a photo detector (e.g., photo sensing device). At 910, an ambient light level is determined based on the detected light level. The ambient light may be from overhead lights, night lights, photo emissions (e.g., photo emission 440 at safe levels) from the PPG sensor 105 itself, and the like. In some examples, as in the case of a toilet lid 110, which blocks out all external lights when it is in a closed position, but resides some distance (e.g., 0.2 inches) above the PPG sensor 105, the toilet lid 110 can be distinguished from tissue that is residing the same distance (e.g., 0.2 inches) above the PPG sensor 105 due to the absorption/reflectance property differences (and the resulting profile of photons) between the manmade material of the toilet lid (e.g., plastic) and tissue. In other words, the resulting profile of the ambient light resulting from the safe photo emissions interacting with the object (i.e., manmade toilet lid 110 vs. tissue) enables the obstruction detection function to identify tissue as an obstruction while identifying the toilet lid 110, as not an obstruction. In such examples, the tissue may be treated as an obstruction while the toilet lid 110 may not be treated as an obstruction.

It is appreciated that the wavelength of the photons (e.g., light) emitted by the photo emitting device when the photo emitting device is in an inactive mode (e.g., safety lockout mode) may be limited to wavelengths that the human eye is not sensitive to and/or is outside the visible spectrum (e.g., infrared wavelengths). For example, in a dark room, the PPG light source may be used at an inactive, low level to excite the photo detector for the purpose of detecting an obstruction in an absence of other ambient light.

At 915, a determination is made (e.g., continually made) as to whether the photo detector is obstructed based on whether the ambient light level satisfies a first threshold. This first threshold may vary based on whether the PPG sensor 105 is emitting any photons at a safe level (e.g., photon emissions 440). In cases, where there is a lack of external lights providing an ambient level of photons (outside of an obstruction), the photo emitting device may emit photons at a safe level to ensure that sufficient ambient light is available to make reliable obstruction detection determinations.

At 920, a PPG light source (e.g., photo emitting device) is operated at a first level (e.g., emitting no photons as illustrated in FIG. 3 or emitting photons 440 at a safe level as illustrated in FIG. 4) when the photo detector is unobstructed. At 925, the PPG light source is operated at a second level (e.g., emitting photons 440-a at a PPG measurement level as illustrated in FIG. 8 that enables the collection of PPG measurements from the tissue) when the photo detector is obstructed. This ensures that photo emissions are within safety thresholds at all times when the PPG sensor 105 is unobstructed, while enabling normal operation when the PPG sensor 105 is appropriately obstructed (e.g., with tissue).

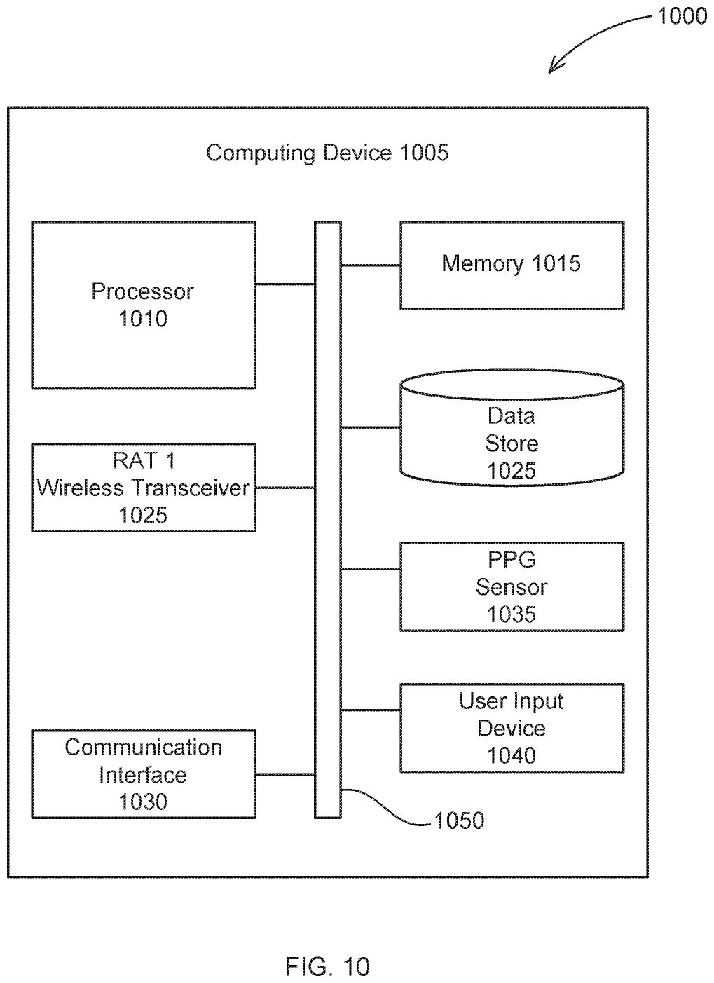

FIG. 10 is a block diagram of a computing device 1005 for implementing the described systems, methods, and devices. In some embodiments, the PPG sensor 105 may be an example of the computing device 1005.

The computing device 1005 includes a processor 1010 (including a general-purpose processor and one or more application specific processors, for example), an optional wireless transceiver 1025 for communicating via a first radio access technology (RAT) (e.g., 3G, 4G, LTE, 5G-NR, and/or LoRaWAN), an optional communication interface 1030 for communicating via a network (e.g., wired, wireless (e.g., Bluetooth, Wi-Fi)), a memory 1015 (e.g., random access memory (RAM), non-volatile RAM (NVRAM)), data store 1020 (e.g., hard disk drive, solid state disk), a PPG sensor 1035 that includes the photo emitting device and the photo sensing device, for example, a user input device 1040 (e.g., touch input, mouse, keyboard, pen input), and an interconnect or bus 1050 for interconnecting each of the components 1010-1040.

In some embodiments, the memory 1015 and/or the data store 1020 (each being a non-transitory storage medium, for example) may store instructions that are executable by the processor 1010 (e.g., processor and memory 1015) to implement the systems, methods, and devices described herein. For example, the instructions may be executable by the processor 1010 to implement any of the methods (e.g., method 900).

The invention has been described with reference to various specific and preferred embodiments and techniques. Nevertheless, it is understood that many variations and modifications may be made while remaining within the spirit and scope of the invention.

What is claimed is:

1. A sensor, comprising:
a photoplethysmography (PPG) light source;
a photo detector;
a memory; and
a processor in electronic communication with the memory stores instructions that when executed by the processor cause the processor to:
detect a light level via the photo detector;
determine an ambient light level based on the detected light level;
determine that the photo detector is obstructed when the ambient level satisfies a first threshold;
operate the PPG light source at a first level when the photo detector is unobstructed; and
operate the PPG light source at a second level when the photo detector is obstructed.

2. The PPG sensor of claim 1, wherein the instructions are further executable by the processor to determine that the photo detector is unobstructed when the ambient light level exceeds the first threshold.

3. The PPG sensor of claim 1, wherein the PPG light source, when operated at the second level, emits photons with sufficient energy to generate PPG signals from human tissue, wherein the PPG signals are generated by the emitted photons that are selectively and characteristically reflected by the human tissue, and wherein the human tissue is leg tissue.

4. The PPG sensor of claim 3, wherein the photo detector receives the PPG signals reflected by the human tissue.

5. The PPG sensor of claim 1, wherein the PPG light source, when operated at the first level, emits no photons, wherein the PPG light source is off when operated at the first level.

6. The PPG sensor of claim 1, wherein the PPG light source, when operated at the first level, emits photons at a safe energy level or emits photons at an unsafe energy level for short enough durations of time to render the emitted photons safe to enter a human eye without causing harm to the human eye, wherein the PPG light source emits photons that are safe for the human eye when operated at the first level.

7. The PPG sensor of claim 6, wherein the PPG light source, when operated at the first level, emits photons that enable self-diagnostic of the PPG sensor, and wherein the PPG light source, when operated at the first level, emits photons that enable calibration of the PPG sensor.

8. The PPG sensor of claim 6, wherein the ambient light is substantially provided by the PPG light source when operated at the first level.

9. The PPG sensor of claim 1, wherein the PPG light source is at least one of a light emitting diode and a laser.

10. The PPG sensor of claim 1, wherein the photo detector is one of a photo diode, a photo transistor, and a photo resistor.

11. A method for operating an unsheathed photoplethysmography (PPG) sensor, comprising:
detecting a light level via a photo detector;
determining an ambient light level based on the detected light level;
determining that the photo detector is obstructed when the ambient light level satisfies a first threshold;
operating a PPG light source at a first level when the photo detector is unobstructed; and
operating the PPG light source at a second level when the photo detector is obstructed.

12. The method of claim 11, further comprising determining that the photo detector is unobstructed when the ambient light level exceeds the first threshold.

13. The method of claim 11, wherein operating the PPG light source at the second level comprises emitting photons with sufficient energy to generate PPG signals from human tissue, wherein the PPG signals are generated by emitting photons that are selectively and characteristically reflected by the human tissue, and wherein the human tissue is leg tissue.

14. The method of claim 13, further comprising receiving PPG signals via the photo detector, wherein the PPG signals are reflected photons emitted by the PPG light source, and wherein the emitted photons are reflected by the human tissue.

15. The method of claim 11, wherein operating the light source at the first level comprises emitting no photons, wherein the PPG light source is off when operating the PPG light source at the first level.

16. The method of claim 11, wherein operating the PPG light source at the first level comprises emitting photons that are safe to enter a human eye without causing harm to the human eye, and wherein emitting photons that are safe to enter the human eye comprises emitting photons at a safe energy level or emitting photons at an unsafe energy level for short enough durations of time to render the emitted photons safe to enter a human eye.

17. The method of claim 16, wherein operating the PPG light source at the first level comprises emitting photons that enable self-diagnostic of the PPG sensor, and wherein operating the PPG light source at the first level comprises emitting photons that enable calibration of the PPG sensor.

18. The method of claim 16, wherein the ambient light level is substantially provided by operating the PPG light source at the first level.

19. The method of claim 11, wherein the PPG light source is at least one of a light emitting diode and a laser.

20. The method of claim 11, wherein the photo detector is one of a photo diode, a photo transistor, and a photo resistor.
